(19) United States
(12) Patent Application Publication
(10) Pub. No.: US 2022/0176117 A1
(43) Pub. Date: Jun. 9, 2022

(54) TRANSCUTANEOUS CURRENT CONTROL APPARATUS AND METHOD

(71) Applicant: BIO-MEDICAL RESEARCH LIMITED, Galway (IE)

(72) Inventors: Conor MINOGUE, Galway (IE); Prasad DESHPANDE, Galway (IE); Daniel COLEMAN, Galway (IE); Glen SHEARER, Galway (IE)

(21) Appl. No.: 17/599,275

(22) PCT Filed: Oct. 2, 2019
(86) PCT No.: PCT/EP2019/076785

§ 371 Date: Sep. 28, 2021

(30) Foreign Application Priority Data
Mar. 29, 2019 (EP) PCT/EP2019/058113

(51) Int. Cl. A61N 1/36; A61N 1/04

(52) U.S. Cl. CPC A61N 1/3603; A61N 1/36034; A61N 1/0408

(57) ABSTRACT

The present invention provides an apparatus and method for limiting the power output of a transcutaneous electrical stimulator in response to changes in electrode impedance. The apparatus comprises pulse generating means having output terminals for delivering a pulsed electrical current through a circuit that contains at least two electrodes intended to be attached to the skin; measuring means coupled to the pulse generator and configured to measure the voltage across the output terminals in response to applied current; comparing means coupled to the measuring means and configured to compare the voltage measured during the pulse against a voltage threshold; and control means coupled to the comparing means and configured to limit the phase charge of the pulse when the measured voltage exceeds the voltage threshold.

### FIELD OF THE INVENTION

The invention relates to a transcutaneous current control apparatus and method. In particular, the invention relates to a transcutaneous current control apparatus and method for use in transcutaneous electrical stimulation.

### BACKGROUND OF THE INVENTION

In transcutaneous electrical stimulation (TES) it is important to achieve a good quality electrical contact with the skin such that the electrical signal is transferred across the skin and into the underlying tissues while avoiding damage to the skin and minimizing any pain or discomfort due to stimulation of pain receptors. Skin electrodes are typically designed to extend over an area of skin ranging between 5 and 200 cm². Passing an electric current through the skin involves a transduction between electron current flow in the wires and metal electrodes of the stimulator system and ionic current flow in the body. This transduction takes place partly through electrolysis and therefore an electrolyte is required at the interface between the metal (or other conductive material) electrode and the skin. It is usually desirable in transcutaneous electrical stimulation that the current density be minimised since this reduces power dissipation per unit area of skin and also reduces the likelihood of stimulating pain receptors in the skin. Normally therefore the electrolyte needs to extend over the full area of the electrode to ensure that the current density into the skin is uniform over the contact surface area. It is also important that the full available area of the electrode makes contact with the skin. If the effective electrode area is reduced, for example due to partial lifting of the electrode from the skin, then the contact area is reduced. When a constant current controlled generator is used, this means the current density in the remaining contact area is increased. This may cause skin irritation, discomfort or pain. The same applies if the electrolyte is distributed unevenly over the area of surface contact, or if the skin is partially covered by grease or dirt.

Increasingly stimulation electrodes are being built into tightly fitting garments or other applicators that are worn by the user, since they are convenient and intuitive to use. There is a particular problem with garment integrated electrical stimulation which relies on pressure instead of adhesive hydrogel to maintain electrical contact with the skin over the area of the electrodes. If the garment is poorly fitting, or the electrode momentarily peels from the skin during movement, the user may experience discomfort as the current is delivered through a reduced area of skin contact causing an increase in current density.

The electrical resistance of the conductors in a garment for electrical stimulation, such as conductive threads, polymers, inks and adhesives, can be much higher than conventional conductors such as copper wire traditionally used in electrical stimulation devices. Furthermore, the resistance of these materials can change with stretch, flexion and age. Washing can affect the resistance of conductors that become exposed to water and detergents. For these reasons it is preferable to use a stimulator which automatically adjusts the output voltage to achieve a predetermined current. Such constant current pulse generators are well known in the field of electronics and may be defined as an electronic control system that adjusts the output voltage to achieve a predetermined current. Preferably the constant current generator operates in the range 0 to 200 mA, or more preferably 0 to 100 mA. A constant current controller does not necessarily mean the current in a waveform is constant with respect to time, rather it means the control system acts to maintain the current at the predetermined value, even if that predetermined value changes with time.

By contrast, a constant voltage stimulator maintains a predetermined voltage waveform and the current is determined by the impedance of the load.

The principal disadvantage of the constant current approach is that it can lead to high current density during electrode peeling and there is a need for systems to protect against this occurrence.

There is therefore a need for a system to detect a peeled electrode very rapidly before a painful or harmful effect occurs. This can be done by reducing the current pulse amplitude and/or phase duration of the stimulation pulse in response to the increased load impedance. Either approach leads to a reduction in phase charge and thereby rms current and therefore current density at the electrode

Systems for measurement of skin contact resistance are well known and may be used to estimate the skin contact resistance of a pair of series connected skin electrodes. It is also well known that the skin-electrode connection can be modelled with the network shown in FIG. 1. The series resistor Rs largely accounts for wiring resistance as well as the resistance of the subcutaneous tissues. The RpCp combination models the impedance of the stratum corneum. The general voltage waveform that results from a constant current pulse has also been well documented and is shown in FIG. 2. Skin contact detection therefore amounts to measuring the resultant voltage across the electrode and comparing with an acceptance threshold. If the threshold is exceeded the system can be programmed to halt the stimulation and notify the user by means of an alarm or other indicator.

There is however considerable variation in how best to define and implement an acceptance criterion for electrode quality.

The U.S. Pat. No. 4,088,141 (Niemi) describes a circuit for monitoring the resistance of an electrode for transcutaneous stimulation. Despite showing the waveform which occurs in response to a current pulse, it is stated that only the initial step voltage V1 is required to assess the electrode quality. Col 3 lines 55 to 68. The decision to terminate the stimulation is based on the leading edge voltage which largely ignores the area of contact. An acceptance threshold set in this way risks many false positives in situations where the series resistance is high but the capacitance is large. Equally, it can fail to detect problems where the series resistance is low but where the electrode area is low leading to small capacitance.

In U.S. Pat. No. 9,474,898 B2 (Gozani et al) a solution is proposed to this problem for a series combination of two electrodes where the impedance measured during the stimulation session is divided by the baseline impedance measured at the start of the session. In this case the impedance is estimated from a “pseudo resistance” which is evaluated at the end of the pulse by dividing the peak voltage by the current. If the impedance ratio to baseline increases beyond an area dependent predefined value then it is assumed that the area of contact of one of the electrodes has reduced below and acceptable level. In this case the acceptance threshold is a ratio of two pseudo resistances evaluated at different time points in a treatment, where the reference resistance is assumed to represent a good contact electrode. A limitation of this approach is that the pseudo resistance would be different with a different pulse width since the peak voltage increases with increased pulse width. Therefore, the baseline and subsequent waveforms have to be the same pulse width. Also, since the evaluation is done at the end of the pulse, the charge has already been delivered by the time the problem is discovered. Although this document mentions altering the stimulus intensity inversely in response to the measured impedance ratio exceeding the acceptance threshold, it is not clear how this altered intensity would be calculated.

It is an object of the invention to obviate or mitigate the above drawbacks.

### SUMMARY OF THE INVENTION

There is therefore a need for an improved approach to changes in electrode impedance during transcutaneous stimulation. One objective of the present invention is to provide an apparatus and method for preventing pain or tissue damage which occurs when the current density exceeds certain limits.

In a first aspect of the present invention there is provided an apparatus for limiting the power output of a transcutaneous electrical stimulator in response to changes in electrode impedance, the apparatus comprising: pulse generating means having output terminals for delivering a pulsed electrical current through a circuit that contains at least two electrodes intended to be attached to the skin; measuring means coupled to the pulse generator and configured to measure the voltage across the output terminals in response to applied current; comparing means coupled to the measuring means and configured to compare the voltage measured during the pulse against a voltage threshold; and control means coupled to the comparing means and configured to limit the phase charge of the pulse when the measured voltage exceeds the voltage threshold.

In one or more embodiments, the pulse generating means is a constant current controlled generator.

In one or more embodiments, the pulse generating means is configured to generate a biphasic current pulse.

In one or more embodiments, each pulse by the pulse generating means has a predetermined phase charge.

In one or more embodiments, each pulse generated by the pulse generating means has a predetermined pulse duration.

In one or more embodiments, the control means is configured to limit a phase charge of a second phase of the pulse to be the same as that of a leading phase of the pulse, even in the situation where the leading phase of the pulse is truncated.

In one or more embodiments, the voltage threshold is constant throughout the pulse and is set to the predicted final voltage for the phase charge and the electrodes in use.

In one or more embodiments, the voltage threshold is updated at time points during the pulse dependent upon the predicted accumulated charge delivered up to each timepoint.

In one or more embodiments, the comparing means comprises a voltage comparator for comparing the measured voltage and the voltage threshold, wherein the voltage comparator is configured to generate an output signal when the measured voltage exceeds the voltage threshold.

In one or more embodiments, the apparatus further comprises converting means for converting the measured voltage to a digital signal.

In one or more embodiments, the comparing means comprises a digital comparator for comparing the digital signal representing the measured voltage with a digital signal representing the voltage threshold, wherein the digital voltage comparator is configured to generate an output signal when the digital signal representing the measured voltage exceeds the digital signal representing the voltage threshold.

In one or more embodiments, the control means is configured to receive the output signal from the comparing means and to determine, based on the output signal, whether to limit the phase charge of the pulse.

In one or more embodiments, the control means comprises software means for detecting a signal outputted from the comparing means, wherein the control means is configured to determine, based on the signal detected by the software means, whether to limit the current amplitude of the pulse.

In one or more embodiments, the control means is configured, based on the output signal from the comparing means. to reduce a voltage available to a constant current circuit.

In one or more embodiments, the voltage threshold is predetermined.

In one or more embodiments, the voltage threshold is determined by analysis of data from multiple users of the same electrode configuration.

In a second aspect of the present invention there is provided a method of limiting the power output of a transcutaneous electrical stimulator in response to changes in electrode impedance, the method comprising: using a pulse generating means having output terminals to deliver a pulsed electrical current through a circuit that contains at least two electrodes intended to be attached to the skin; measuring the voltage across the output terminals in response to applied current; comparing the voltage measured during the pulse against a voltage threshold; and limiting the phase charge of the pulse when the measured voltage exceeds the voltage threshold.

### BRIEF DESCRIPTION OF THE DRAWINGS

Embodiments of the present invention are described hereinafter with reference to the accompanying drawings in which:

FIG. 1 shows an equivalent circuit for transcutaneous electrical stimulation;

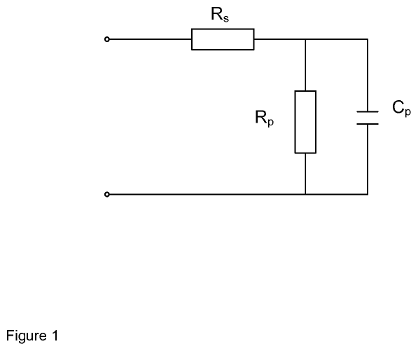

FIG. 2 shows a typical voltage and current waveforms during a constant current pulse;

FIG. 3 a) depicts a typical symmetric biphasic square current waveform with an interphase interval. b) is a monophasic current waveform with the same current amplitude;

FIG. 4 shows a circuit schematic of a battery operated electrical stimulator based on the current invention;

FIG. 5 shows a further embodiment of a circuit where a comparator is used to control the duration of stimulation pulses though an AND gate;

FIG. 6 shows an illustration of a train of current pulses and the associated voltage pulses under 4 different load conditions;

FIG. 7 shows actual voltage waveforms recorded from several users at the same current level. A voltage limit is also shown which changes during the course of the pulse;

FIG. 8 shows actual voltage waveforms from a single user across a range of current amplitudes; and

FIG. 9 shows computed voltage waveforms based on a published model of a 50 cm2 electrode. (Vargas Luna, Krenn et al. 2015)

### DETAILED DESCRIPTION OF THE INVENTION

Pulse train characteristics/power and current limits.

A pulse in the present context may be defined as a time limited current flow in an electrical circuit. The duration for which the current flows is called the pulse width and is typically in the range 10 to 1000 μs, though pulses lasting several milliseconds are also used in electrical stimulation. Usually pulses are produced sequentially as a train of pulses and the number of pulses per second is known as the frequency. A pulse may be characterised in terms of the amplitude of voltage or current that arises while the current is flowing. Often a pulse is described by a waveform which is a graphical representation of how the current or voltage varies with time during the course of the pulse train. The direction of flow of current in the circuit is given by the phase of the waveform, a monophasic waveform comprises a sequence of pulses which flow in the same direction. A biphasic pulse contains two phases where the direction of current flow is reversed between phases. In this case the pulse width is defined as sum of each phase duration plus the interval between them.

Pulses can have a rectangular shape which means that the current is at a fixed amplitude during the pulse. Pulses can also be triangular, ramp, exponential or half-sine shapes, meaning that the predetermined amplitude is intended to vary according to these functions.

The frequency, pulse width, phase durations, and amplitude are usually predetermined in a treatment regime. For each pulse that is issued the intended phase duration, pulse width, and current amplitude are preset in the stimulator at the start of each pulse. (Usually it is the user that presets the amplitude and may alter it as the treatment progresses). The pulse that is actually delivered, however depends on the load and the limitations of the hardware. For example, if the intended current was 50 mA for 400 μs, a charge of 20 μC, into a load of 1500Ω, the stimulator might not be capable of delivering such a charge into the load at the required pulse frequency.

TES devices normally use relatively low frequency pulse trains (0 to 150 Hz), with pulse durations in the range 100 to 1000 μs. The pulses can be monophasic or biphasic with amplitudes ranging up to 200 mA but more usually less than 100 mA. Because the duty cycle is typically low, the root mean square (rms) current is usually much less than the peak current. Tissue damage is believed to occur when the power density exceeds 0.25 W/cm2. The safety standard ECC 60601-2-10 requires that the user's attention be drawn to situations where the current density can exceed rms 2 mA/cm2. We believe that user comfort is best protected by restricting the current density to 1 mA/cm2, or more preferably rms 0.5 mA/cm2.

The maximum rms current of a typical TES device for muscle stimulation range from 10 to 30 mA (rms) per channel. A typical electrode might be as low as 25 cm2, so it is easy to see how electrode peeling can give rise to pain. Typical electrodes use adhesive hydrogel to keep them secured to the skin.

Painful sensation can arise with a sustained current density in excess of about 0.5 mA rms/cm². However, a single pulse having a peak current density of 2 mA/cm2 can readily be tolerated. A momentary increase in current density, lasting a few hundred microseconds, does not cause a painful stimulus or temperature rise. A burst of such pulses would cause discomfort, more so if the frequency is higher, because the rms current is increased. If an electrode is subject to partial peeling during movement but reconnects more fully soon afterwards, it is desirable the stimulation not stop but rather that the rms current reduces when the impedance increases and is restored when the impedance recovers. Thus pain and discomfort can be controlled if the rms current is reduced in proportion to the impedance at the electrodes. This occurs quite naturally in a constant voltage stimulator because increased impedance results in a reduced current. However, in a constant current stimulator the drive circuit compensates for a higher impedance by applying a higher voltage. If the increased electrode impedance is due to a reduced electrode area of contact, then maintaining a constant current results in an increased current density.

There is therefore a need to control the rms current in a constant current pulsed transcutaneous stimulation device so as to prevent pain and discomfort.

The root mean square current of a periodic waveform is

$${i}_{rms} = \sqrt{\frac{1}{T} {\int }_{0}^{T} {i ( t )}^{2} dt}$$

Where i(t) is the current as a function of time, T is the period of the periodic waveform. The average power dissipated in a load R is then

P_{avg}=iᵣₘₛ²R

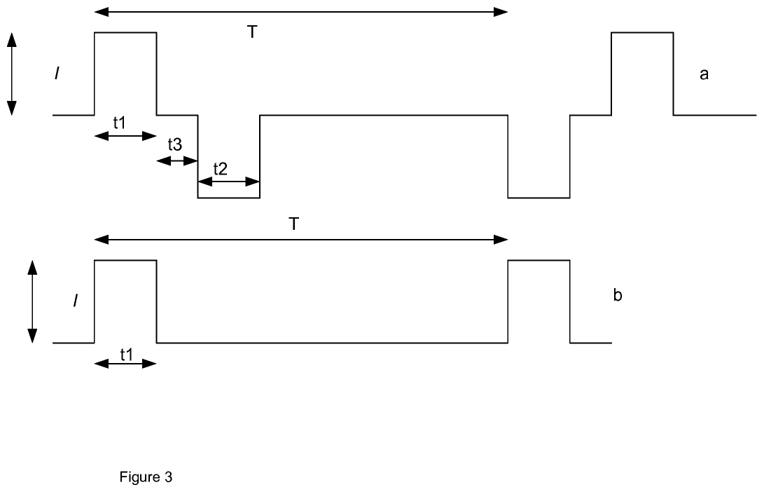

Most NMES and TENS devices provide a pulsed current, where the duration of the pulse is much shorter than the interval between the pulses. (see FIG. 3)

The period of each waveform is T and is typically much longer then the phase duration t1 or t2. The frequency of the waveform is the inverse of T.

For a square wave such as at FIG. 3b the rms current calculation simplifies to

$${i}_{rms} = I \sqrt{\frac{t 1}{T}}$$

Or, for a typical symmetric biphasic waveform like that at FIG. 3a it would be

$${i}_{rms} = I \sqrt{\frac{t 1 + t 2}{T}}$$

The rms current can therefore be adjusted by controlling either or all of the variables I, t1, t2 (if applicable) and T.

The present invention provides a means to control t1 and t2 dynamically such that the rms current is regulated in response to changes in the load impedance.

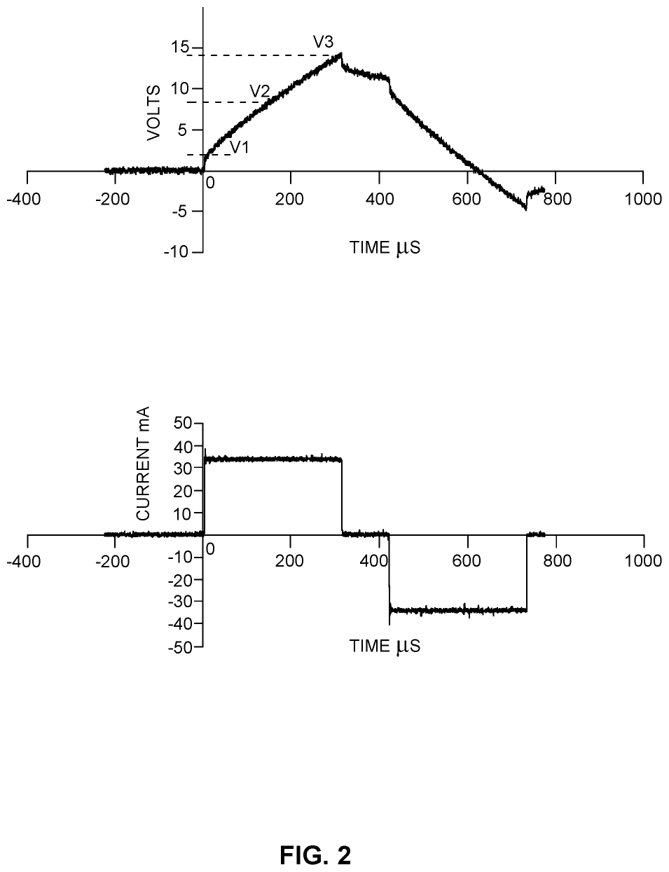

The typical voltage characteristic across the electrodes in response to a constant current pulse is shown in FIG. 2. There is a step increase in voltage at the start of the current pulse followed by a more gradual but steady increase in voltage as the capacitance in the electrode-skin interface charges. The rate of increase depends on the capacitance as well as the current applied. If the capacitance decreases, such as when an electrode in the garment peels off, then the rate of change of voltage increases. The present invention sets a limit on the electrode voltage and terminates the current when the voltage exceeds that limit. It does not stop the stimulation entirely. It limits the current applied by curtailing the pulse width to the moment at which the electrode voltage exceeded the limit. A normal pulse interval follows before another current pulse is initiated. Each pulse may be truncated at the point at which the electrode exceeded the voltage limit for the pulse

The firmware determines the voltage limit by combining a number of factors. The first factor is the expected charge that is delivered to the electrode. In one embodiment it is the expected electrical charge to be delivered by the completed pulse, which for a square wave current is the product of the preset current amplitude and the preset phase duration. Their product amounts to a charge in microcoulombs. An alternative embodiment estimates the charge delivered at any point within the pulse by simply integrating the expected current.

The second factor relates to the area of the electrode pair being used which in effect determines the expected electrode capacitance and shunt resistance. We have determined this factor empirically through measurement of the voltage across the electrode as the area of contact is adjusted for a number of subjects. Alternatively, this factor can be determined by reference to published models of electrode impedance such as that by Vargas (Vargas Luna, Krenn et al. 2015)

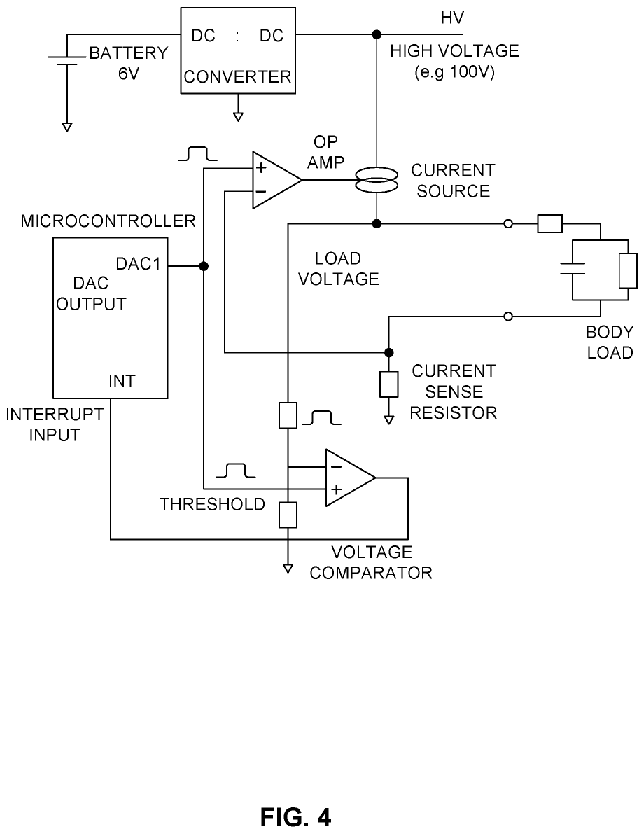

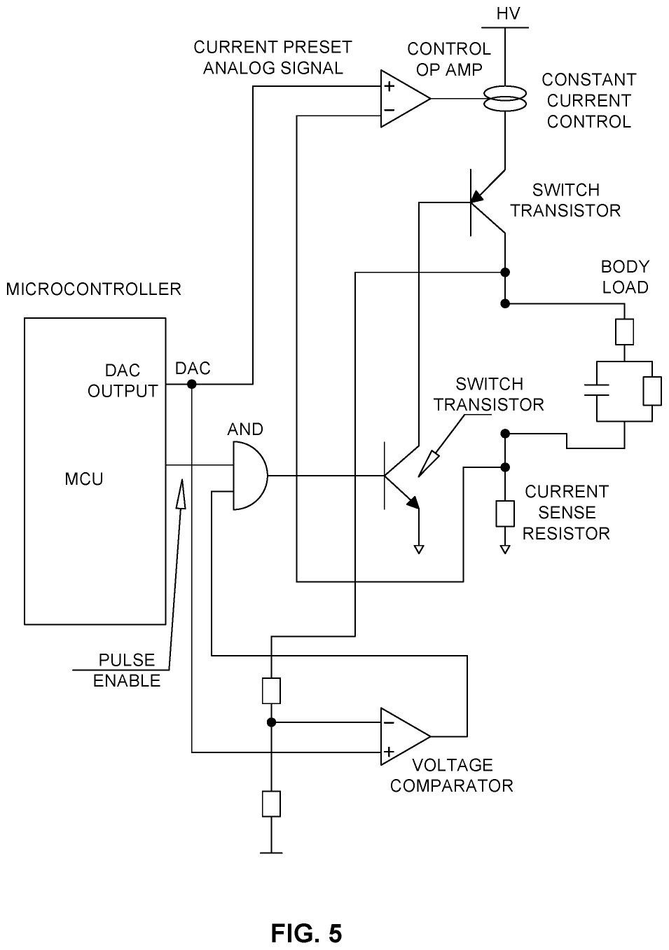

There are a number of ways to accomplish this control of phase duration. FIG. 4 shows a simple schematic diagram for a battery operated electrical stimulator for producing monophasic pulses. Biphasic pulses can be produced by including a H-bridge circuit as is well known in the art. It has a DC:DC converter to create a high voltage source. A microcontroller generates a timed pulse of controllable amplitude through a digital to analog converter. This feeds a constant current generator which produces a current pulse into the load. A simple voltage comparator is provided which has as one of its inputs a signal representing the voltage across the selected electrodes. The other comparator input is provided with a reference that is synthesized through a digital to analog converter from the micro-controller. The output of the comparator is fed back to the microcontroller. If the actual voltage exceeds the reference voltage at any time during the pulse then the microcontroller can react quickly to terminate the pulse, effectively reducing the phase duration of the pulse. Alternatively, the comparison can be made by digitising the electrode voltage direction in an analog to digital converter and comparing it with the reference in a digital comparator within the microcontroller or otherwise within the firmware. Either method also allows the firmware to quickly terminate the pulse if required. In both of these methods the reference value is adjusted by the firmware based on the selected current and phase duration. In this way the electrode check is reduced to sampling a voltage and comparing with a pre-calculated reference value. This enables very fast detection of an electrode problem and minimal delay in terminating the pulse. A further example is shown in FIG. 5 where the comparator output is used to gate the stimulation pulse. There are various other circuit variations which could be used to achieve this effect the essential aspect of which is to modulate the phase duration in response to the voltage across the load.

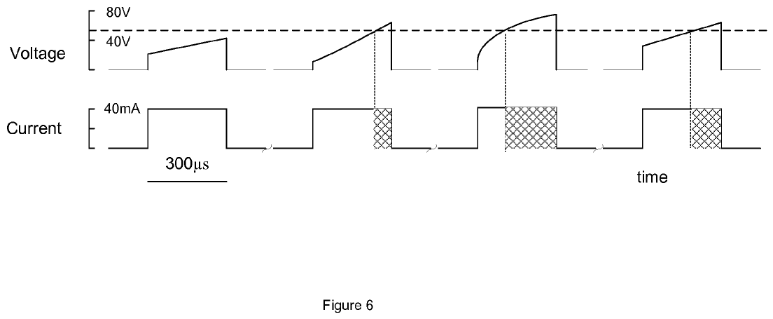

An illustration of the effect is shown in FIG. 6 where the dotted line in the upper voltage trace shows the threshold level set by the microcontroller based on the expected current, phase duration, and electrode type. In pulse 1 a full pulse is delivered because the voltage did not exceed the threshold. The subsequent pulses are each truncated at the moment the voltage exceeded the threshold. The resultant pulse has a reduced charge and the pulse train has a reduced rms current and therefore a reduced current density.

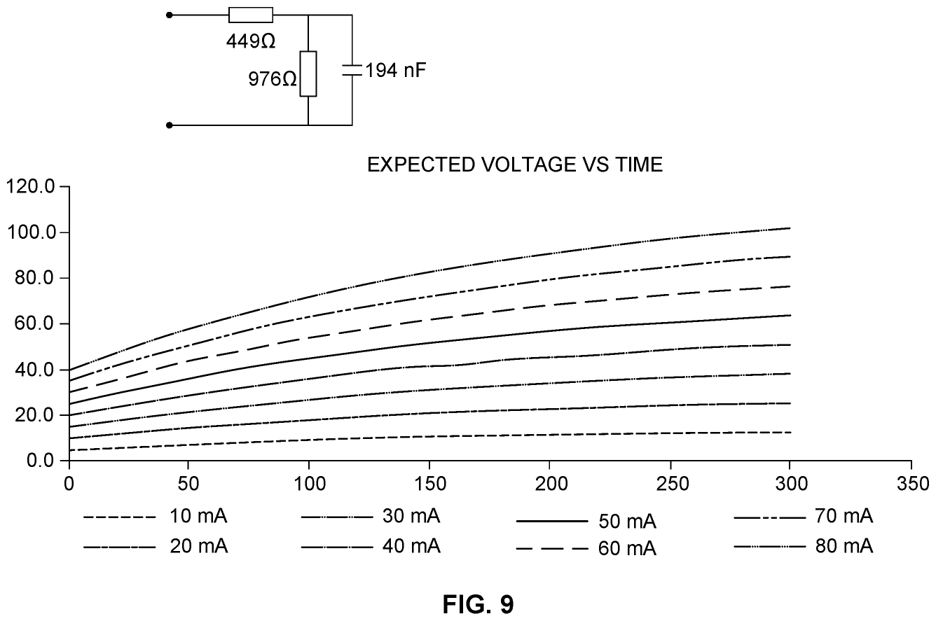

The selection of the threshold voltage limit is critical to achieving a satisfactory result. There can be false positive reactions if the level is set too low for an individual, or conversely, the system may fail to reduce the current density if the threshold level is set too high. The ideal voltage is that which is just necessary to deliver the expected charge with full electrode contact and no more. FIG. 9 shows a family of curves representing the expected voltage that would arise during constant current square pulse of 300 μS duration, across a range of current levels into a typical load reported from literature for a 50 cm² electrode. The voltage threshold to the comparator can be selected to be the voltage at the end of the pulse. A reduction in electrode area associated with peeling has the effect of reducing the electrode capacitance thereby causing an increased rate of change of voltage such that the threshold is reached earlier in the pulse. The pulse can be terminated at this point, so limiting the rms current and consequently the current density delivered to the load.

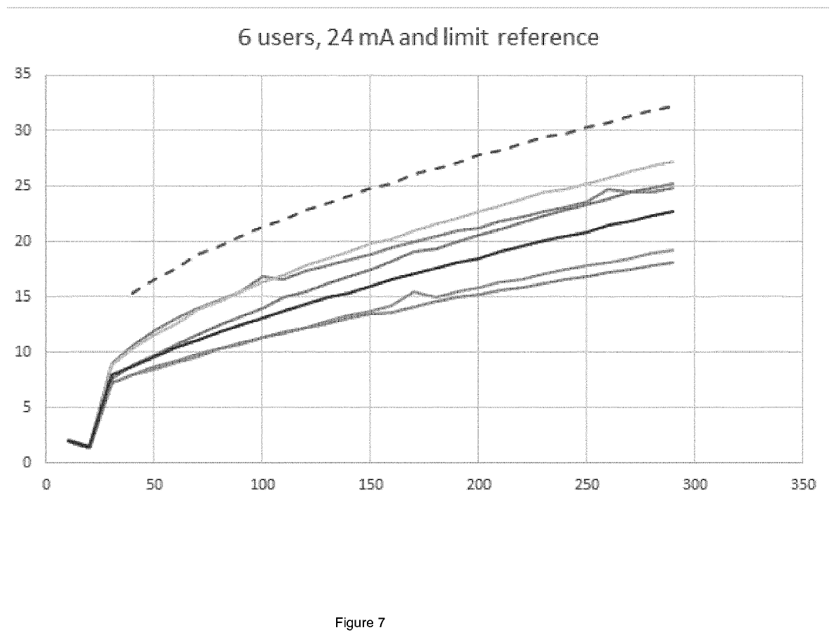

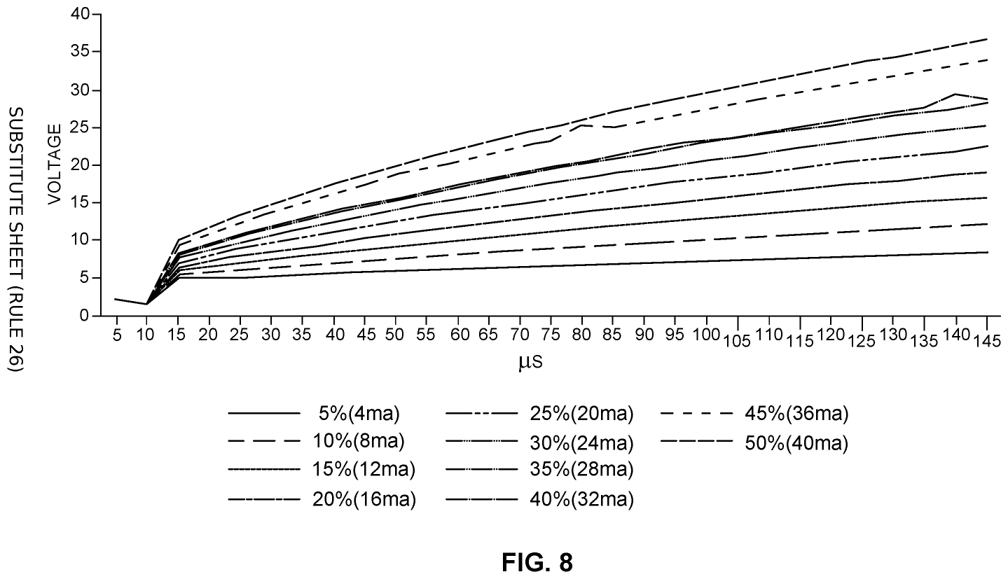

The expected voltage for a given electrode design can also be derived empirically through experimentation with users to derive a family of curves at different current amplitudes. Such a family of curves for a single user is shown in FIG. 8 for a range of currents. The variation between users at the same current level is illustrated in FIG. 7 showing that the expected voltage and thereby the threshold value, for a given charge delivered, varies between people and can even vary within people between different sessions. It is possible to define a threshold limit based on the group mean and 95% confidence interval of the mean. The threshold could be set to the upper end of the confidence interval initially. It is envisaged that this invention is particularly relevant where electrodes are incorporated within garments or other body-worn applicators and as such will be used by the same user. It is therefore possible to employ machine learning techniques that monitor the voltage during the stimulation pulse to arrive at improved estimates of the expected voltage limits to be applied for a range of current amplitude and phase durations for a given user. These estimates can be stored between treatments to improve the accuracy of the system over time.

Modern stimulators are often connected to the internet and so data from many users can be collated to gain statistical information about electrode voltages for a large population. This allows further optimisation of the acceptance limits for specific electrode configurations and even user characteristics such as gender and BMI.

The expected voltage may also be referred to as the predicted voltage. The threshold reference is in effect a predicted voltage that is based on the characteristics of the current pulse to be delivered and a model of the load.

This adaptive technique can be improved by getting an input from the user as to the comfort of the stimulation. This can be easily arranged by seeking a comfort score from the user through the user interface, during the treatment or after the session is completed. This score could be from a visual analog scale input through a smartphone app touchscreen connected to the stimulator. Such an input allows the system to correlate the measured voltage with a comfort level.

In a further embodiment of the invention the output is regulated by simply limiting the voltage available to the constant current circuit, thereby reducing the amplitude of the current. For example, the circuit of FIG. 4 could be adapted to allow the microcontroller to set the voltage of the DC:DC converter, or using a voltage amplifier which allows the microcontroller to regulate the supply voltage available at the output of the DC:DC converter before connection to the current controller.

We have performed extensive testing of electrodes within garments, where there is moderate pressure applied and where the skin has been wetted with saline. We have found that electrodes that are intended to have a skin contact area of greater than 60 cm², can be comfortably peeled off across a range of currents if the voltage limit is set to the expected voltage at the end of the pulse for that current and phase duration.

It is very important in transcutaneous stimulation have a balanced current waveform such that there is little or no DC current through the skin since otherwise unwanted electrolytic effects can occur with skin irritation and even damage. If, according to the present invention, the leading phase of a biphasic waveform is truncated then it is important that the second or trailing phase is truncated to the same time, or that the overall charge transferred is otherwise balanced with the leading phase. A simple way of doing this in FIG. 4 is for the microcontroller record the exact duration of the leading phase and use this data to control the duration of the second phase.

The voltage threshold can be adjusted during the pulse to improve the sensitivity of the current control mechanism. In FIG. 7 a voltage limit is shown which is initially low and increases steadily throughout the pulse. The initial level is much lower than the final expected voltage and therefore is able to detect electrode faults earlier in the pulse, for example, where the electrode has insufficient electrolyte and therefore has a high value of Rs leading to a higher initial step voltage.

Modifications are possible within the scope of the invention, the invention being defined in the appended claims.

### REFERENCES

Vargas Luna, J. L., M. Krenn, J. A. Cortes Ramirez and W. Mayr (2015). “Dynamic impedance model of the skin-electrode interface for transcutaneous electrical stimulation.” PLoS One 10(5): e0125609.

1. An apparatus for limiting the power output of a transcutaneous electrical stimulator in response to changes in electrode impedance, the apparatus comprising:
pulse generating means having output terminals for delivering a pulsed electrical current through a circuit that contains at least two electrodes intended to be attached to the skin;
measuring means coupled to the pulse generator and configured to measure the voltage across the output terminals in response to applied current;
comparing means coupled to the measuring means and configured to compare the voltage measured during the pulse against a voltage threshold; and
control means coupled to the comparing means and configured to limit the phase charge of the pulse when the measured voltage exceeds the voltage threshold.

2. The apparatus of claim 1, wherein the pulse generating means is a constant current controlled generator.

3. The apparatus of claim 1, wherein pulse generating means is configured to generate a biphasic current pulse.

4. The apparatus of claim 1, wherein each pulse by the pulse generating means has a predetermined phase charge.

5. The apparatus of claim 1, wherein each pulse generated by the pulse generating means has a predetermined pulse duration.

6. The apparatus of claim 3, wherein the control means is configured to limit a phase charge of a second phase of the pulse to be the same as that of a leading phase of the pulse, even in the situation where the leading phase of the pulse is truncated.

7. The apparatus of claim 1, wherein the voltage threshold is constant throughout the pulse and is set to the predicted final voltage for the phase charge and the electrodes in use.

8. The apparatus of claim 1, wherein the voltage threshold is updated at time points during the pulse dependent upon the predicted accumulated charge delivered up to each timepoint.

9. The apparatus of claim 1, wherein the comparing means comprises a voltage comparator for comparing the measured voltage and the voltage threshold, wherein the voltage comparator is configured to generate an output signal when the measured voltage exceeds the voltage threshold.

10. The apparatus of claim 1, further comprising converting means for converting the measured voltage to a digital signal.

11. The apparatus of claim 10, wherein the comparing means comprises a digital comparator for comparing the digital signal representing the measured voltage with a digital signal representing the voltage threshold, wherein the digital voltage comparator is configured to generate an output signal when the digital signal representing the measured voltage exceeds the digital signal representing the voltage threshold.

12. The apparatus of claim 9, wherein the control means is configured to receive the output signal from the comparing means and to determine, based on the output signal, whether to limit the phase charge of the pulse.

13. The apparatus of claim 1, wherein the control means comprises software means for detecting a signal outputted from the comparing means, wherein the control means is configured to determine, based on the signal detected by the software means, whether to limit the current amplitude of the pulse.

14. The apparatus of claim 1, wherein the control means is configured, based on the output signal from the comparing means. to reduce a voltage available to a constant current circuit.

15. The apparatus of claim 1, wherein the voltage threshold is predetermined.

16. The apparatus of claim 1, wherein the voltage threshold is determined by analysis of data from multiple users of the same electrode configuration.

17. A method of limiting the power output of a transcutaneous electrical stimulator in response to changes in electrode impedance, the method comprising:
using a pulse generating means having output terminals to deliver a pulsed electrical current through a circuit that contains at least two electrodes intended to be attached to the skin;
measuring the voltage across the output terminals in response to applied current;
comparing the voltage measured during the pulse against a voltage threshold; and
limiting the phase charge of the pulse when the measured voltage exceeds the voltage threshold.
